(19) United States
(12) Patent Application Publication
(10) Pub. No.: US 2001/0001098 A1
(43) Pub. Date: May 10, 2001

(54) Hepatitis B core antigen nucleic acid vaccine

(75) Inventors: Shan Lu, Northborough, MA (US); Zuhu Huang, Nanjing (CN); John E. Herrmann, Northborough, MA (US)

Correspondence Address: J, PETER FASSE, Fish & Richardson P.C., 225 Franklin Street, Boston, MA, 02110-2804, US

(73) Assignee: University of Massachusetts, Massachusetts Corporation

(21) Appl. No.: 09/756,500

(22) Filed: Jan. 8, 2001

Related U.S. Application Data
(63) Continuation of application No. 09/400,497, filed on Sep. 21, 1999, now abandoned.
(60) Provisional application No. 60/101,311, filed on Sep. 21, 1998.

(51) Int. Cl. A61K 31/70; A01N 43/04; A61K 48/00; A01N 63/00

(52) U.S. Cl. 514/44; 424/93.21

(57) ABSTRACT

Hepatitis B virus core antigen nucleic acid vaccines and their use are disclosed. In the vaccines and methods of the invention, precore sequences in the 5′ untranslated region of the core antigen mRNA are not present.

### CROSS REFERENCE TO RELATED APPLICATION

1. This application claims priority from U.S. provisional application Ser. No. 60/101,311, filed on Sep. 21, 1998, which is incorporated herein by reference in its entirety.

### FIELD OF THE INVENTION

2. This invention relates to virology and immunology.

### BACKGROUND OF THE INVENTION

3. Hepatitis B virus (HBV) chronically infects liver tissue in humans, with the highest prevalence of infection in Asia. HBV infection has been correlated with liver cirrhosis, liver failure, and liver cancer.

4. HBV-infected individuals often produce an immune response to the major viral nucleocapsid protein, the hepatitis B viral core antigen (HBcAg). HBcAg is encoded by the viral pre-C/C gene, which transcribes a long and short mRNA. The long mRNA contains a first AUG, beginning the coding sequence for the precore polypeptide, and a second AUG downstream and inframe with the first, beginning the coding sequence for HBcAg. The short mRNA contains only the second AUG encoding HBcAg and a precore 5′ untranslated region (UTR). Thus, the polypeptide translated from the long mRNA contains the HBcAg polypeptide sequence with a N-terminal precore amino acid sequence. Transcription of the long and short mRNA is regulated during the viral life cycle.

### SUMMARY OF THE INVENTION

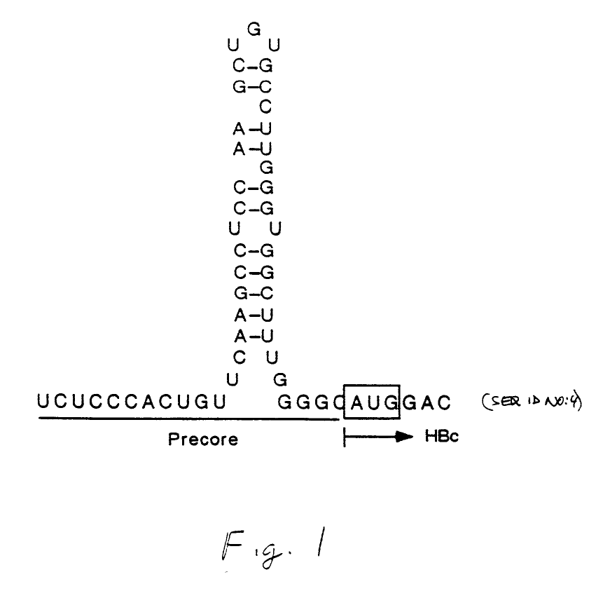

5. It has been discovered that removal of the viral precore 5′ UTR sequence in a mammalian expression vector encodingHBcAg results in a surprisingly effective HBcAg nucleic acid vaccine. One reason for the enhanced effectiveness of the HBcAg nucleic acid vaccine appears to be the removal of a stem-loop structure in the 35 nucleotides immediately upstream of the HBcAg start codon (FIG. 1).

6. Accordingly, the invention features a method of eliciting an immune response against a hepatitis B virus in a mammal, e.g., a human. The method includes providing a composition containing an isolated nucleic acid which includes: (1) a nucleotide sequence encoding a HBcAg polypeptide, (2) a start codon immediately upstream of the nucleotide sequence encoding the HBcAg polypeptide, (3) a mammalian promoter operably linked to the nucleotide sequence, and (4) a mammalian polyadenylation signal operably linked to the nucleotide sequence. The promoter directs transcription of a mRNA encoding the HBcAg polypeptide, and the 35 nucleotides immediately upstream of the HBcAg start codon (in the 5′ untranslated region of the mRNA) are free of the sequence:

7. aagccuccaagcugugccuuggguggcu (SEQ ID NO:1).

8. The entire 5′ untranslated region of the mRNA can be free from SEQ ID NO:1. The composition is administered to the mammal so that the HBcAg protein is expressed in the mammal at a level sufficient to elicit an immune response against the hepatitis B virus.

9. The invention also features a composition containing an isolated nucleic acid which includes: (a) a nucleotide sequence encoding a HBcAg polypeptide, (b) a start codon immediately upstream of the nucleotide sequence, (c) a mammalian promoter operably linked to the nucleotide sequence, and (d) a mammalian polyadenylation signal operably linked to the nucleotide sequence. The promoter directs transcription of a mRNA encoding the HBcAg, and the 35 nucleotides immediately upstream of the start codon for HBcAg in the 5′ untranslated region of the mRNA are free of SEQ ID NO:1. In some embodiments, the entire 5′ untranslated region of the mRNA is free of SEQ ID NO:1.

10. A suitable mammalian promoter is a cytomegalovirus immediate-early promoter. The promoter can include a cytomegalovirus intron A operably linked to the mammalian promoter. A suitable mammalian polyadenylation signal can be derived from a bovine growth hormone gene.

11. In some embodiments, a composition or method of the invention elicits a serum anti-HBcAg antibody level of at least 20 Paul-Ehrlich Institute (PEI) units per milliliter (e.g., greater than 30, 40, or 100 PEI units/ml) in the mammal.

12. A composition containing the nucleic acid can include a pharmaceutically acceptable carrier, an adjuvant, or particles that bind to the nucleic acid. Particles in the composition are advantageous when the route of administration is by particle bombardment of the skin or a mucosal surface of the mammal. In other embodiments, the composition is administered by intramuscular injection. In some embodiments, a method of the invention includes repeated administration of the composition.

13. The invention also features a method of eliciting an immune response against a hepatitis B virus core antigen (HBcAg) in a mammal by providing a composition having an isolated nucleic acid which includes: (a) a nucleotide sequence encoding a HBcAg polypeptide, (b) a start codon immediately upstream of the sequence coding the HBcAg polypeptide, (c) a mammalian promoter operably linked to the nucleotide sequence, and (d) a mammalian polyadenylation signal operably linked to the nucleotide sequence. The composition is administered to the mammal so that the nucleic acid expresses the HBcAg polypeptide in the mammal at a level sufficient to produce a serum anti-HBcAg antibody level of at least 20 PEI units per milliliter.

14. A “nucleic acid vaccine” is a vaccine whose active ingredient is at least one isolated nucleic acid which encodes a polypeptide antigen.

15. An “isolated nucleic acid” is a nucleic acid free of the genes that flank the gene of interest in the genome of the organism or virus in which the gene of interest naturally occurs. The term therefore includes a recombinant DNA incorporated into an autonomously replicating plasmid. It also includes a separate molecule such as a cDNA, a genomic fragment, a fragment produced by polymerase chain reaction, or a restriction fragment. It also includes a recombinant nucleotide sequence that is part of a hybrid gene, i.e., a gene encoding a fusion protein. An isolated nucleic acid is substantially free of other cellular or viral material (e.g., free from the protein components of a viral vector), or culture medium when produced by recombinant techniques, or substantially free of chemical precursors or other chemicals when chemically synthesized.

16. Expression control sequences are “operably linked” when they are incorporated into other nucleic acid so that they effectively control expression of a gene of interest.

17. As used herein, “protein” or “polypeptide” means any peptide-linked chain of amino acids, regardless of length or post-translational modification, e.g., glycosylation or phosphorylation.

18. As used herein, a “Paul-Ehrlich Institute unit” or “PEI unit” is a unit of anti-HBcAg antibody titer equal to that defined by the reference anti-HBcAg antibody standard available from the Paul-Ehrlich Institute (Langen, Germany).

19. An “adjuvant” is a compound or mixture of compounds which enhances the ability of a nucleic acid vaccine to elicit an immune response.

20. A “mammalian promoter” is any nucleic acid sequence, regardless of origin, that is capable of driving transcription of a mRNA coding for a HBcAg within a mammalian cell.

21. A “mammalian polyadenylation signal” is any nucleic acid sequence, regardless of origin, that is capable of terminating transcription of an mRNA encoding an HBcAg within a mammalian cell.

22. Unless otherwise defined, all technical and scientific terms used herein have the same meaning as commonly understood by one of ordinary skill in the art to which this invention belongs. Although suitable methods and materials for the practice or testing of the present invention are described below, other methods and materials similar or equivalent to those described herein, which are well known in the art, can also be used. All publications, patent applications, patents, and other references mentioned herein are incorporated by reference in their entirety. In case of conflict, the present specification, including definitions, will control. In addition, the materials, methods, and examples are illustrative only and not intended to be limiting.

23. Other features and advantages of the invention will be apparent from the following detailed description, and from the claims.

### BRIEF DESCRIPTION OF THE DRAWING

24. FIG. 1 is a schematic diagram illustrating the predicted stem-loop structure in the precore sequence immediately upstream of the HBcAg start codon (SEQ ID NO:4).

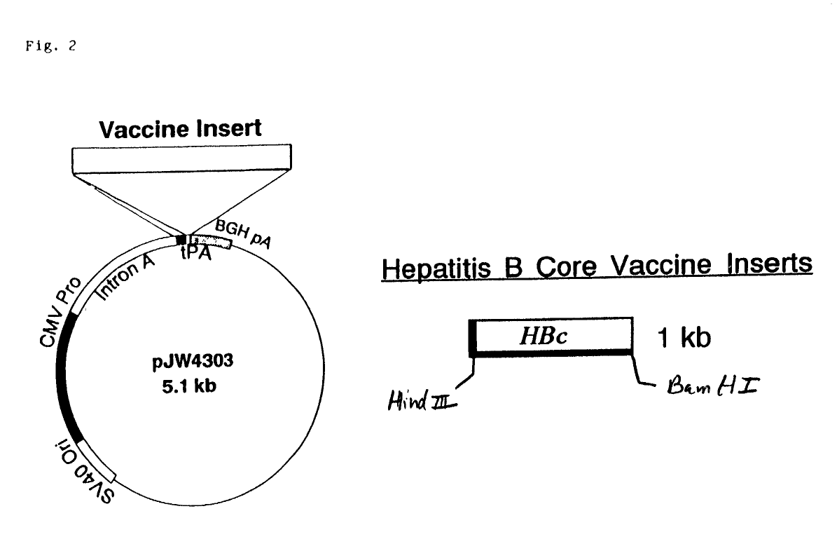

25. FIG. 2 is a schematic diagram illustrating the cloning strategy used to generate the HBcAg expression vector.

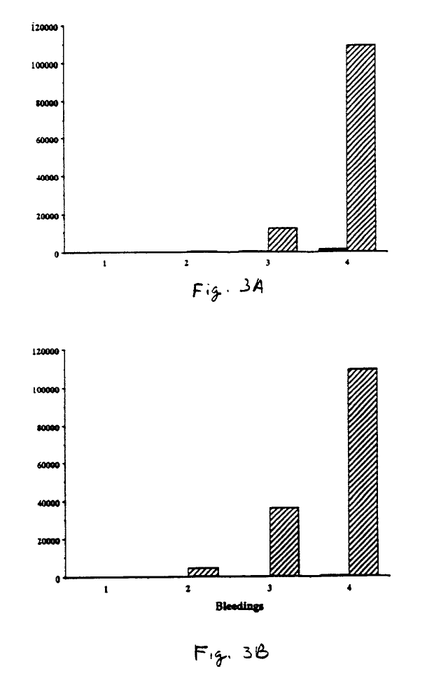

26. FIGS. 3A and 3B are bar graphs of anti-HBcAg antibody levels in mice immunized by intramuscular injection of a HBcAg nucleic acid vaccine. Immunized Balb/c mice are represented in FIG. 3A. Immunized C57/BL6 mice are represented in FIG. 3B. The solid bars are the serum titer for mice receiving pJW4303. The hatched bars are the serum titer for mice receiving pJW4303/HBc. Bleedings 1-4 represent samples taken at 0, 4, 8, and 12 weeks after the first immunization, respectively.

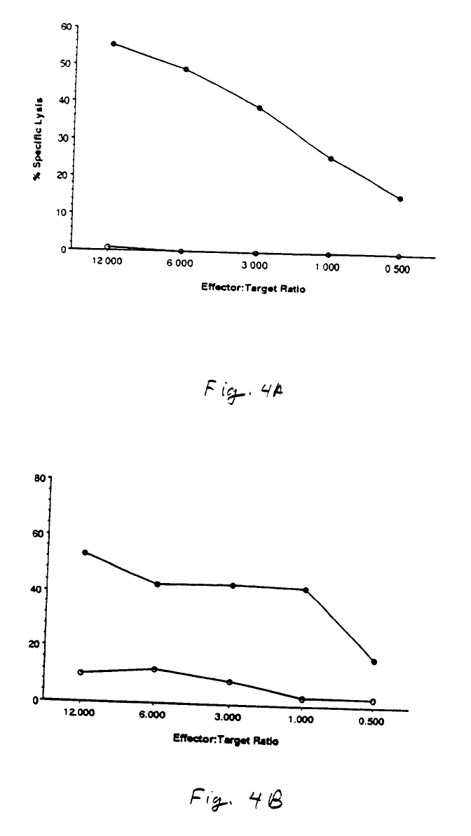

27. FIGS. 4A and 4B are graphs of cytotoxic T-cell responses in mice immunized by intramuscular injection of a HBcAg nucleic acid vaccine. Immunized Balb/c mice are represented in FIG. 4A. Immunized C57/BL6 mice are represented in FIG. 4B. Solid circles represent specific lysis for mice receiving pJW4303. Open circles represent specific lysis for mice receiving pJW4303/HBc.

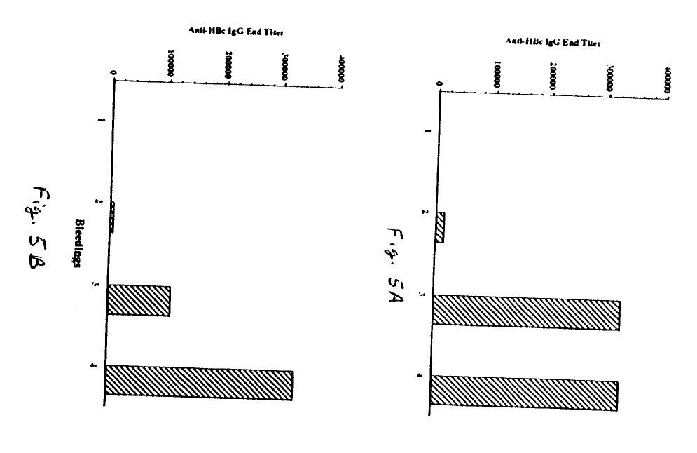

28. FIGS. 5A and 5B are bar graphs of anti-HBcAg antibody levels in mice immunized with a HBcAg nucleic acid vaccine by particle bombardment. Immunized Balb/c mice are represented in FIG. 5A. Immunized C57/BL6 mice are represented in FIG. 5B. The solid bars are the serum titer for mice receiving pJW4303. Hatched bars represent serum titer for mice receiving pJW4303/HBc. Bleedings 1-4 represent samples taken at 0, 4, 8, and 12 weeks after the first immunization, respectively.

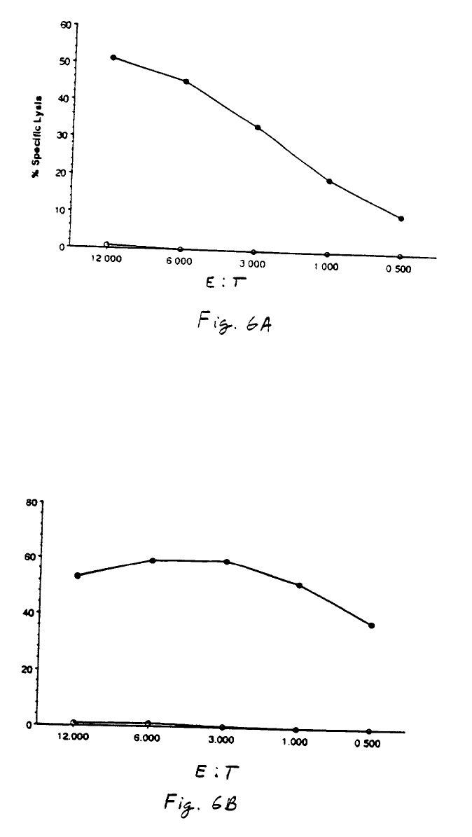

29. FIGS. 6A and 6B are graphs of cytotoxic T-cell responses in mice immunized with a HBcAg nucleic acid vaccine by particle bombardment. Balb/c mice were immunized in FIG. 6A, and C57/BL6 mice were immunized in FIG. 6B. The solid circles are the specific lysis for mice receiving pJW4303. The open circles are the specific lysis for mice receiving pJW4303/HBc.

### DETAILED DESCRIPTION

30. An effective HBV DNA vaccine provides advantages over a protein subunit vaccine because DNA is stable under a variety of conditions. This allows for ease in storage and shipping, especially in lesser developed countries. Because the vaccine need not contain an adjuvant (see Example I below), raw material costs and manufacturing costs are lower. Like HBV subunit vaccines, HBV DNA vaccines are safer than vaccines based on live vectors such as viruses or bacteria. Additional advantages include the production of a more native antigen conformation, ease of modifying the amino acid sequence of the antigen, and ability to co-deliver nucleic acids that can express other antigens or polypeptide adjuvants (e.g., cytokines).

31. The nucleic acid vaccines of the invention can be used as prophylactic vaccines in naive individuals, or as therapeutic vaccines in individuals already infected with HBV.

32. Nucleic Acids Encoding HBcAg Polypeptides

33. An HBcAg polypeptide encoded by a nucleic acid used in the methods or compositions of the invention is any protein or polypeptide sharing an epitope with a naturally occurring HBcAg. Such functionally related HBcAg polypeptides can differ from the wild type HBcAg sequence by additions or substitutions within the HBcAg amino acid sequence. Amino acid substitutions may be made on the basis of similarity in polarity, charge, solubility, hydrophobicity, hydrophilicity, and/or the amphipathic nature of the residues involved.

34. Nonpolar (hydrophobic) amino acids include alanine, leucine, isoleucine, valine, proline, phenylalanine, tryptophan, and methionine. Polar neutral amino acids include glycine, serine, threonine, cysteine, tyrosine, asparagine, and glutamine. Positively charged (basic) amino acids include arginine, lysine, and histidine. Negatively charged (acidic) amino acids include aspartic acid and glutamic acid.

35. HBcAg variants with altered amino acid sequences can be obtained by random mutations to HBcAg DNA (See U.S. Pat. No. 5,620,896). This can be achieved by random mutagenesis techniques known in the art. Following expression of the mutagenized DNA, the encoded polypeptide can be isolated to yield highly antigenic HBcAg. Alternatively, site-directed mutations of the HBcAg coding sequence can be engineered using techniques also well-known to those skilled in the art.

36. In designing variant HBcAg polypeptides, it is useful to distinguish between conserved positions and variable positions. To produce variants with increased antigenicity, conserved residues preferably are not altered. Alteration of non-conserved residues are preferably conservative alterations, e.g., a basic amino acid is replaced by a different basic amino acid. Similar mutations to the HBcAg coding sequence can be made to generate HBcAg polypeptides that are better suited for expression in vivo.

37. The nucleic acids useful in the methods and compositions of the invention include at least three components: (1) a HBcAg coding sequence beginning with a start codon, (2) a mammalian transcriptional promoter operatively linked to the coding sequence for expression of the HBcAg, and (3) a mammalian polyadenylation signal operably linked to the coding sequence to terminate transcription driven by the promoter. In this context, a “mammalian” promoter or polyadenylation signal is not necessarily a nucleic acid sequence derived from a mammal. For example, it is known that mammalian promoters and polyadenylation signals can be derived from viruses.

38. In addition, complete HBcAg nucleic acid sequences are known. See, e.g., Pasek et al., Nature 282:575-579 (1979), which discloses a sequence available under GenBank Accession No. J02202.

39. The nucleic acid vector can optionally include additional sequences such as enhancer elements, splicing signals, termination and polyadenylation signals, viral replicons, and bacterial plasmid sequences. Such vectors can be produced by methods known in the art. For example, nucleic acid encoding the desired HBcAg can be inserted into various commercially available expression vectors. See, e.g., Invitrogen Catalog, 1998. In addition, vectors specifically constructed for nucleic acid vaccines are described in Yasutomi et al., J Virol 70:678-681 (1996).

40. Administration of Nucleic Acids

41. The nucleic acids of the invention can be administered to an individual, or inoculated, in the presence of substances that have the capability of promoting nucleic acid uptake or recruiting immune system cells to the site of the inoculation. For example, nucleic acid encapsulated in microparticles have been shown to promote expression of rotaviral proteins from nucleic acid vectors in viva (U.S. Pat. No. 5,620,896).

42. A mammal can be inoculated with nucleic acid through any parenteral route, e.g., intravenous, intraperitoneal, intradermal, subcutaneous, intrapulmonary, or intramuscular routes. It can also be administered, orally, or by particle bombardment using a gene gun. Muscle is a useful tissue for the delivery and expression of HBcAg-encoding nucleic acid because mammals have a proportionately large muscle mass which is conveniently accessed by direct injection through the skin. A comparatively large dose of nucleic acid can be deposited into muscle by multiple and/or repetitive injections. Multiple injections can be used for therapy over extended periods of time.

43. Administration of nucleic acids by conventional particle bombardment can be used to deliver nucleic acid for expression of HBcAg in skin or on an mucosal surface. Particle bombardment can be carried out using commercial devices. For example, the Accell IIυ (PowderJect Vaccines, Inc., Middleton, Wis.) particle bombardment device, one of several commercially available “gene guns”, can be employed to deliver nucleic acid-coated gold beads. A Helios Gene Gun (Bio-Rad) can also be used to administer the DNA particles. Information on particle bombardment devices and methods can be found in sources including the following: Yang et al., Proc Natl Acad Sci USA 87:9568 [1990]; Yang, CRC Crit Rev Biotechnol 12:335 [1992]; Richmond et al., Virology 230:265-274 [1997]; Mustafa et al., Virology 229:269-278 (1997); Livingston et al., Infect Immun 66:322-329 (1998) and Cheng et al., Proc Natl Acad Sci USA 90:4455 [1993].

44. In some embodiments of the invention, an individual is inoculated by a mucosal route. The HBcAg-encoding nucleic acid can be administered to a mucosal surface by a variety of methods including nucleic acid-containing nose- drops, inhalants, suppositories, or microspheres. Alternatively, a nucleic acid vector containing the HBcAg gene can be encapsulated in poly(lactide-co-glycolide) (PLG) microparticles by a solvent extraction technique, such as the ones described in Jones et al., Infect Immun 64:489 (1996); and Jones et al., Vaccine 15:814 (1997). For example, the nucleic acid is emulsified with PLG dissolved in dichloromethane, and this water-in-oil emulsion is emulsified with aqueous polyvinyl alcohol (an emulsion stabilizer) to form a (water-in-oil)-in-water double emulsion. This double emulsion is added to a large quantity of water to dissipate the dichloromethane, which results in the microdroplets hardening to form microparticles. These microdroplets or microparticles are harvested by centrifugation, washed several times to remove the polyvinyl alcohol and residual solvent, and finally lyophilized. The microparticles containing nucleic acid have a mean diameter of 0.5 μm. To test for nucleic acid content, the microparticles are dissolved in 0.1 M NaOH at 100° C. for 10 minutes. The A₂₆₀ is measured, and the amount of nucleic acid calculated from a standard curve. Incorporation of nucleic acid into microparticles is in the range of 1.76 g to 2.7 g nucleic acid per milligram PLG.

45. Microparticles containing about 1 to 100 μg of nucleic acid are suspended in about 0.1 to 1 ml of 0.1 M sodium bicarbonate, pH 8.5, and orally administered to mice or humans, e.g., by gavage.

46. Regardless of the route of administration, an adjuvant can be administered before, during, or after administration of the nucleic acid. An adjuvant can increase the uptake of the nucleic acid into the cells, increase the expression of the antigen from the nucleic acid within the cell, induce antigen presenting cells to infiltrate the region of tissue where the antigen is being expressed, or increase the antigen-specific response provided by lymphocytes.

47. Evaluating Vaccine Efficacy

48. Before administering the vaccines of this invention to humans, efficacy testing can be conducted using animals. In an example of efficacy testing, mice are vaccinated by intramuscular injection. After the initial vaccination or after optional booster vaccinations, the mice (and negative controls) are monitored for indications of vaccine-induced, HBcAg-specific immune responses. Methods of measuring HBcAg-specific immune responses are described in the Examples below and also in Townsend et al., J Virol 71:3365-3374 (1997); Kuhober et al., J Immunol 156: 3687-3695 (1996); Kuhrober et al., Int Immunol 9:1203-1212 (1997); Geissler et al., Gastroenterology 112:1307-1320 (1997); and Sallberg et al., J Virol 71:5295-5303 (1997).

49. Anti-HBcAg serum antibody levels in vaccinated animals can be determined using the CORE anti-HBc kit (cat. no. 2259-20, Abbott GmbH, Weisbaden, Germany). The concentrations of anti-HBcAg antibodies are standardized against a readily available reference standard of the Paul-Ehrlich Institute (Langen, Germany).

50. Cytotoxicity assays can be performed as follows. Spleen cells from immunized mice are suspended in complete MEM with 10% fetal calf serum and 5×10⁻⁵ M 2-mercapto-ethanol. Cytotoxic effector lymphocyte populations are harvested after 5 days of culture, and a 5-hour ⁵¹Cr release assay is performed in a 96-well round-bottom plate using target cells. The effector to target cell ratio is varied. Percent lysis is defined as (experimental release minus spontaneous release) / (maximum release minus spontaneous release)×100.

### EXAMPLES

51. The invention is further illustrated by the following examples. The examples are provided for illustration only, and are not to be construed as limiting the scope or content of the invention in any way.

### Example 1: Administration of HBcAg Nucleic Acid by Intramuscular Injection into Mice

52. To construct an expression vector for use as the HBcAg nucleic acid vaccine, two plasmids were used (FIG. 2). The pJW4303 plasmid containing a cytomegalovirus immediate-early promoter with intron A and a bovine growth hormone polyadenylation signal was described in Yasutomi et al., J Virol 70:678-681 (1996). The fragment containing the HBcAg -coding sequence was derived from plasmid pYTA1, which was described in Yie et al., Chinese J Virol 4:312-318 (1988). The HindIII-BamHI fragment of pYTA1 included the full coding sequence of HBcAg without any precore viral sequences upstream of the HBcAg start codon. The HBcAg nucleic acid vaccine vector was generated by inserting the HindIII-BamHI fragment of pYTA1 into the HindIII and BamHI sites in the polylinker of pJW4303, the polylinker being just downstream of the cytomegalovirus intron A in pJW4303. The new vector was designated pJW4303/HBc.

53. To test for expression of HBcAg from the new plasmid, pJW4303/HBc was used to transfect 293T cells. 48 hours after transfection, the cell lysates were harvested and subjected to ELISA and Western blotting. Transient expression of HBcAg in 293T cells was clearly demonstrated by both methods.

54. After confirming that the pJW4303/HBc drove expression of HBcAg in vitro, the plasmid was used to vaccinate mice by intramuscular injection. A total of 100 μg of pJW4303/HBc in saline was bilaterally injected into the quadriceps muscles of a BALB/c mouse and a C57BL/6 mouse. A second BALB/c mouse and C57BL/6 mouse received 100 μg of pJW4303 in like fashion as controls. The mice were supplied by Taconic Farms, Inc. and maintained in the animal colony facility of the University of Massachusetts Medical Center. All mice were 6-8 weeks old females at the time of the first inoculation. The injections were repeated at 4 and 8 weeks after the first inoculation.

55. At 0, 4, 8, and 12 weeks after the initial immunization (Bleedings 1-4, respectively), serum samples were taken from all four mice. End-point titration of anti-HBc antibodies was performed by ELISA using a microtiter plate coated with recombinant HBcAg protein (0.1 μg/well). Three-fold serially diluted serum samples were incubated in the coated wells for 30 minutes. The liquid was then removed, and the wells washed. The wells were then incubated with biotinylated goat anti-mouse IgG for 30 minutes, followed by washing. Streptavidin-linked horseradish peroxidase (HRP, Vector laboratories, Inc.) was then added and incubated for 30 minutes, followed by washing. HRP substrate 3,3′,5,5′-tetramethylbenzidine (TMB) was then added to the wells to develop color, and the amount of converted substrate was read in a microplate reader.

56. As shown in FIG. 3A, the anti-HBcAg antibody levels in the Balb/c mouse receiving pJW4303/HBc (hatched bars) was above that of the Balb/c mouse receiving the control plasmid (solid bars) by the third bleeding. As shown in FIG. 3B, the anti-HBcAg antibody levels in the C57/BL6 mouse receiving the pJW4303/HBc plasmid was above that of the control mouse by the second bleeding. The titers determined by ELISA were confirmed using the COREZYME kit (Abbott), which was standardized against the serum standard available from the Paul-Ehrlich Institute. It was determined that the titer of about 150,000 by the fourth bleeding in HBcAg immunized mice represented an unexpected titer of at least about 500 PEI units/ml. Previous publications have described anti-HBcAg antibody responses of no more than 10 PEI units/ml in animals receiving a HBcAg nucleic acid vaccine (Kuhober et al., J Immunol 156: 3687-3695 [1996] and Kuhrober et al., Int Immunol 9:1203-1212 [1997]).

57. To determine if any cytotoxic T cell response against HBcAg was generated in immunized mice, the mice were sacrificed at 12 weeks after the third inoculation. Single spleen cell suspensions were prepared. Cytotoxic effector lymphocyte populations were harvested after 6 days of culture and resuspended at 1×10⁶ cells/ml. A 4-hour ⁵¹Cr release assay was performed in a 96-well round-bottom plate using P815 cells (H-2^{d}-restrictive, for Balb/c mice) or EL-4 cells (H-2^{b} restrictive, for C57/BL6 mice) as the target cells. The synthesized H-2^{d}-restricted peptide SYVNTNMGL, (SEQ ID NO:2) was added to the Balb/c spleen cell reaction at 10 μg/ml, and the synthesized H-2^{b}-restricted peptide (MGLKFRQL; SEQ ID NO:3) was added to the C57/BL6 spleen cell reaction, also at 10 μg/ml. The effector cell to target cell (E:T) ratios used were 12:1, 6:1, 3:1, 1:1, and 0.5:1. Percent lysis was defined as (experimental release-spontaneous release) / (maximum release-spontaneous release)×100.

58. As shown in FIG. 4A, at least 50% specific lysis could be achieved by an E:T ratio of above 6:1 in Balb/c mice vaccinated with pJW4303/HBc. A similar immune response was observed in the C57/BL6 mice. As shown in FIG. 4B, at least 50% specific lysis could be achieved by an E:T ratio of 12:1 in mice vaccinated with pJW4303/HBc. Thus, the pJW4303/HBc nucleic acid vaccine, without adjuvants, elicited both significant antibody and cell-mediated immune responses in animals.

### Example 2: Administration of HBcAg Nucleic Acid by Particle Bombardment

59. To test another route of administration, the pJW4303/HBc and the pJW4303 control DNA was delivered intradermally by particle bombardment. The Accell II™ particle bombardment device (Powderject Vaccines, Inc., Middleton, Wis.) was employed to deliver DNA-coated gold beads to the epidermis of two Balb/c and two C57/BL6 mice, one of each pair receiving the HBcAg plasmid and the other of each pair receiving the control DNA.

60. For delivery by particle bombardment, DNA was precipitated onto 0.95 or 1- to 3-μm gold beads (Degussa, South Plainfield, N.J.) with 100 mM spermidine and 2.5 M CaCl₂ at 1 μg of DNA per 0.5 mg gold shot (Eisenbraun et al., DNA Cell Biol 12:791-797 [1993]).

61. Mice were anesthetized with 30 μl of Ketaset/Rompun (10:2). Abdominal target areas were shaved and thoroughly rinsed with water prior to gene delivery. Nucleic acid-coated gold particles were delivered into abdominal skin with the Accell II™ gene gun, which employed a helium discharge as the motive force. Each animal received six nonoverlapping deliveries per immunization, each delivery at 300-400 pounds per square inch. The immunization was repeated at 4 weeks and 8 weeks after the first immunization.

62. Antibody and cytotoxic T cell responses were determined as described in Example 1 above. As shown in FIG. 5A, the immunization elicited an antibody titer of over 300,000 (corresponding to at least about 1000 PEI units/ml) in a Balb/c mouse by the third bleeding. Again, like the intramuscular results in Example 1, this antibody response was unexpectedly high as compared to previous studies. The antibody response elicited in the C57/BL6 mouse was comparable to that for the Balb/c mice (FIG. 5B). As shown in FIGS. 6A and 6B, the cytotoxic responses in the two strains of mice receiving pJW4303/HBc were similar to that for the intramuscular results described in Example 1 above. Greater than 50% specific lysis was observed at an E:T ratio of 12 for both strains of mice.

63. These results indicated that the HBcAg nucleic acid vaccine described herein produced humoral and cell-mediated immune responses by a variety administration methods.

### Example 3: Intramuscular Injection of HBcAg Nucleic Acid into Monkeys

64. The expression vector (pJW4303/HBc) described in Examples 1 and 2 was also used as an HBcAg nucleic acid vaccine to vaccinate monkeys. After confirming that the pJW4303/HBc vector drove expression of HBcAg in vitro, the plasmid was used to vaccinate monkeys by intramuscular injection.

65. Monkeys in group I (animals #1 and #2) were immunized with HBcAg nucleic acid vaccine while the other two monkeys in group II (#3 and #4) received control plasmid DNA vector without the HBc insert. Each animal received 2.0 mg of DNA plasmids intramuscularly (IM) at each inoculation (delivered equally as 500 μg shots at four muscle sites). The DNA inoculations were given every two months. Animal sera were collected prior to each inoculation and ELISA was done to detect anti-HBV antibody responses.

66. Table 1 below shows antibody responses induced by the HBcAg nucleic acid vaccine in monkeys. Monkeys immunized with the HBcAg nucleic vaccine (#1 and #2) clearly had hepatitis B core specific antibody responses after one immunization (animal #1) or two immunizations (animal #2), respectively. Two negative control monkeys (animal #3 and #4) had no antibody responses against the hepatitis B core antigen. In Table 1 (+) indicates a positive antibody response for hepatitis B core antigen, while (−) means a negative antibody response for hepatitis B core antigen. N/D indicates a test was not done. Animal #1 died of unrelated diseases before sample collection at the 4th month.

TABLE 1
Human hepatitis B core specific antibody response (ELISA)

| | | 0 month | | |
|---|---|---|---|---|
| Monkey | Plasmid used | (prebleed) | 2 months | 4 months |
| #1 | HBcAg DNA vaccine | (−) | (+) | N/D |
| #2 | HBcAg DNA vaccine | (−) | (−) | (+) |
| #3 | Control vector | (−) | (−) | (−) |
| #4 | Control vector | (−) | (−) | (−) |

67. Because it is more difficult to induce immune response by IM DNA immunization in primates, the HBcAg nucleic acid vaccine demonstrated its highly efficient potential to be developed as a clinical vaccine for human use.

### Other Embodiments

68. Other embodiments are within the following claims.

We claim:

1. A method of eliciting an immune response against a hepatitis B virus in a mammal, the method comprising:
obtaining a composition comprising an isolated nucleic acid comprising (a) a nucleotide sequence encoding a hepatitis B virus core antigen (HBcAg) polypeptide, (b) a start codon immediately upstream of the nucleotide sequence, (c) a mammalian promoter operably linked to the nucleotide sequence, and (d) a mammalian polyadenylation signal operably linked to the nucleotide sequence, wherein the promoter directs transcription of a mRNA encoding the HBcAg polypeptide, and wherein the 35 nucleotides immediately upstream of the start codon for HBcAg in the 5′ untranslated region of the mRNA are free of SEQ ID NO:1; and
administering to the mammal the composition in an amount sufficient for the nucleic acid to express the HBcAg protein in the mammal at a level sufficient to elicit an immune response against the hepatitis B virus.

2. The method of claim 1, wherein the mammalian promoter is a cytomegalovirus immediate-early promoter.

3. The method of claim 2, wherein the isolated nucleic acid further comprises a cytomegalovirus intron A operably linked to the mammalian promoter.

4. The method of claim 1, wherein the mammalian polyadenylation signal is derived from a bovine growth hormone gene.

5. The method of claim 1, wherein the entire 5′ untranslated region of the mRNA is free of SEQ ID NO:1.

6. The method of claim 1, wherein the immune response is production of an anti-HBcAg antibody in the serum at a level of at least 20 PEI units per milliliter.

7. The method of claim 1, wherein the composition further comprises a pharmaceutically acceptable carrier.

8. The method of claim 1, wherein the composition further comprises an adjuvant.

9. The method of claim 1, wherein the composition is administered by intramuscular injection.

10. The method of claim 1, wherein the composition further comprises particles to which the nucleic acid is bound, and wherein the composition is administered by particle bombardment of the skin or mucosal surface of the mammal.

11. The method of claim 1, further comprising repeating the administration.

12. The method of claim 1, wherein the animal is a human.

13. A composition comprising an isolated nucleic acid comprising (a) a nucleotide sequence encoding a hepatitis B virus core antigen (HBcAg) polypeptide, (b) a start codon immediately upstream of the nucleotide sequence, (c) a mammalian promoter operably linked to the nucleotide sequence, and (d) a mammalian polyadenylation signal operably linked to the nucleotide sequence, wherein the promoter directs transcription of a mRNA encoding the HBcAg polypeptide, and wherein the 35 nucleotides immediately upstream of the start codon for HBcAg in the 5′ untranslated region of the mRNA are free of SEQ ID NO:1.

14. The composition of claim 13, further comprising an adjuvant.

15. The composition of claim 13, wherein the mammalian promoter is a cytomegalovirus immediate-early promoter.

16. The composition of claim 15, wherein the isolated nucleic acid further comprises a cytomegalovirus intron A operably linked to the mammalian promoter.

17. The composition of claim 13, wherein the mammalian polyadenylation signal is derived from a bovine growth hormone gene.

18. The composition of claim 13, wherein the entire 5′ untranslated region of the mRNA is free of SEQ ID NO:1.

19. The composition of claim 13, further comprising a pharmaceutically acceptable carrier.

20. The composition of claim 13, further comprising particles to which the isolated nucleic acid is bound, wherein the particles are suitable for bombardment of mammalian skin or mucosal surfaces as a form of administration of the isolated nucleic acid.

21. A method of eliciting an immune response against a hepatitis B virus core antigen (HBcAg) in a mammal, the method comprising:
providing a composition comprising an isolated nucleic acid comprising (a) a nucleotide sequence encoding a hepatitis B virus core antigen (HBcAg) polypeptide, (b) a start codon immediately upstream of the nucleotide sequence, (c) a mammalian promoter operably linked to the nucleotide sequence, and (d) a mammalian polyadenylation signal operably linked to the nucleotide sequence; and
administering to the mammal the composition, wherein the nucleic acid expresses the HBcAg polypeptide in the mammal at a level sufficient to elicit a serum anti-HBcAg antibody level of at least 20 PEI units per milliliter.
